(19) United States
(12) Patent Application Publication
(10) Pub. No.: US 2016/0296406 A1
(43) Pub. Date: Oct. 13, 2016

(54) TREATMENT DEVICE FOR STRETCHING THE CARPAL TUNNEL OF THE HUMAN HAND

(71) Applicant: Dieter HEYL, Achern (DE)

(72) Inventor: Dieter Heyl, Achern (DE)

(21) Appl. No.: 15/100,829

(22) PCT Filed: Dec. 2, 2014
(86) PCT No.: PCT/EP2014/076213

§ 371 Date: Jun. 1, 2016

(30) Foreign Application Priority Data
Dec. 5, 2013 (DE) 10 2013 113 564.1

(51) Int. Cl. A61H 1/02; A61H 9/00; A61F 5/32; A61F 5/01; A61F 5/34

(52) U.S. Cl. CPC A61H 1/0285; A61F 5/0118; A61F 5/012; A61F 5/34; A61F 5/32; A61H 9/005; A61H 2205/065

(57) ABSTRACT

The invention relates to a treatment device for stretching the carpal tunnel of the human hand, characterized by at least two adhesive elements, which are suitable to come into contact with human skin and to be applied to the ball of the thumb of a human hand to be treated, and characterized by a stretching device, which is designed to act on the adhesive elements and/or to be connected to the latter and to exert a substantially laterally directed stretching force on the adhesive elements.

### BACKGROUND

The present invention relates to a therapeutic device to stretch the carpal tunnel of the human hand.

The carpal channel, often called the carpal tunnel, is a passageway surrounded by a connective tissue starting from the forearm to the hand on the palmar side of the wrist. The “base” and the side walls of the channel are built by the carpal bone. The “top” of the channel forms the 4-5 cm wide carpal ligament which arches over the carpal bones. Several flexor tendons pass through the channel as well as the median nerve, regulating amongst others the movement of the finger and the thumb also securing their tactile sensation ability. The narrowing of the carpal channel for example due to arthrosis or overstressed swelling conditions can damage the median nerve. This type of damage is called carpal tunnel syndrome and can provoke paraesthesia, numbness, pain and/or movement disorders depending on its progress.

A treatment of the carpal tunnel syndrome often takes place by means of an operation where the carpal ligament is cut through in order to lower the pressure on the median nerve making the area around it more spacious. Apart from the operation there are conservative therapy options such as the wearing of special splints or support bandages. As an additional treatment alternative, special types of splints were suggested, opening the carpal channel by stretching it and thus lengthening the transverse ligaments so that more space is being gained.

In document U.S. Pat. No. 6,146,347 such a splint for stretching the carpal channel is shown. This splint is put around the hand expanding the carpal channel by means of two inflatable air cushions which are located on the back of the hand. The forces being put on the carpal ligament with this splint are relatively low, restricting the field of application of this device.

Another device to stretch the carpal channel is described in document U.S. 2004/0210169. It has an annular mechanism being put around an affected hand. This mechanism exerts pressure on the ball of the thumb as well as on the back of the hand by means of appropriate pads. However, this device is elaborately put together in a relatively complicated way thereby enhancing its production costs. In addition this apparatus is very heavy making its handling uncomfortable. Moreover, in order to achieve higher expansion forces, the vertical force transmission has to be quite high, leading to pain after a short time.

In document U.S. Pat. No. 6,315,748 a bandage is shown, which is put over the back of the hand. This bandage has elastic, self-adhesive tapes that are taped on the palm. However, this bandage does not allow continuous force transmission, as the adhesive tapes are inflexibly affixed. In addition, a suitable counteracting force is missing in order to effectively deform the carpal ligament.

### SUMMARY

Therefore, it is an object of the present invention to provide a therapeutic device for expanding the carpal channel of a human hand, which offers an improved, more precise and effective transmission of the forces needed for the expansion.

The object is achieved by the features of the invention. Advantageous further embodiments can be taken from the description that follows and the claims.

The therapeutic device according to the invention comprises at least two adhesive elements, being convenient concerning direct contact with human skin and being designed for application on the thenar and hypothenar of the affected hand. Further to this, an expansion mechanism is provided which is designed to interfere with the adhesive elements and/or to get or be connected to them and to exert a basically laterally directed expansion force on the adhesive elements.

For treatment, the adhesive elements can be affixed on both sides of the carpal channel, i.e. on the thumb side (thenar) as well as on the hand edge side (hypothenar) eminence of the hand. The laterally directed expansion force is applied on the adhesive elements by means of the expansion mechanism is precisely transferred directly on the thenar and hypothenar eminances on both sides of the carpal channel.

The adhesive elements are preferably provided with straps having at least a self-adhesive coat on one side. If these straps on the opposite sides are furnished with a reversible quick release fastener, preferably a Hook-and-loop fastener (Velcro™), the expansion mechanism can be affixed and removed in a particularly simple manner.

The adhesive elements can be designed in the form of adhesive pads, which are adapted in size and/or shape to the eminences of a human hand to be treated. Different sizes of adhesive pads can also be kept in stock for different hand sizes, from which stock a choice can then be made in the particular treatment case.

In a first embodiment, the expansion mechanism comprises two shoes, which can be spread against one another. For example, the spreading can be effected with a threaded spindle expanding the shoes against each other. Therefore the expansion force can be easily adjusted to the needs of the user respectively patient. The possibility to individually regulate the expansion force is very advantageous, as the sensation of pain of the user can be different and fluctuant in time.

Due to the simple set-up, the expansion mechanism can be made in a very small and compact design, enabling the user to carry it along and to use it whenever needed.

Above the threaded spindle, the shoes can be interconnected at least with one joint. This upper joint produces a counter pressure, which results in that the expansion force being horizontally transferred within the plane of the palm to the expandable carpal ligament.

At the bottom of the expansion mechanism the shoes show a contact surface with a reversible fastener, preferably of Velcro™. Thus, the expansion mechanism can be designed in an easily detachable version.

Preferably the outline of the contact surface of the shoes is individually adjusted to the ball of the thumb of the affected hand. This enables a further improvement of the force transmission. For instance these areas can be individually produced according to a mold taken of the affected hand. Of course anatomically shaped seating areas can be pre-produced for different characteristical hand shapes and hand sizes allowing an individual selection for the customer.

An alternative version of the treatment unit has as expansion mechanism a preferably elastic stretch belt, which is constructed in such a way that it can be put around the back of the affected hand in order to execute the expansion force on the adhesive elements. By means of an expansion mechanism constructed as bandage, a defined counterforce is induced on the back of the hand which will be transferred via the expansion tape on the adhesive elements. In fact the transferable force by means of such an expansion tape is minor compared to the first version, but can be effective over a longer period. Such a bandage is suitable for permanent use, because it does not restrict the user in his mobility.

In order to induce the counterforce on the back of the hand, a pad can be fixed in the middle of the expansion tape. The pad can be particularly an elastic pressure pad, for example a foam body. Alternatively and/or additionally the pad can contain an air cushion, which is inflatable by means of a manual pump.

In case on the internal side of the expansion tape reversible fasteners, preferably Velcro™ fasteners are provided, the expansion tape can be removed very fast. In addition, the expansion force can be easily adjusted by this type of fasteners. Moreover, it is possible to provide various different expansion tapes with different elastic characteristics, offering the possibility to choose the most appropriate one for the adaptation of the individually suitable expansion force.

Such an expansion bandage can be produced very cost-efficiently in large quantities and in different sizes, so that time and effort for individual tailor-made solutions do not apply. A treatment unit like this is ideal for self-treatment of patients being affected.

### BRIEF DESCRIPTION OF THE DRAWINGS

In the following, more advantages and features of the actual invention will be explained by means of figures and examples of execution.

Shown here:

FIG. 1 a human hand with fitted adhesive elements according to the invention.

FIG. 2A a top view on an expansion unit in an initial design example.

FIG. 2B a buttock line through the expansion unit of the initial design example.

FIG. 3 a buttock line through an expansion unit designed as bandage in a second sample.

The solution as per the invention is based on the expansion of the carpal ligament by means of a transmission of a laterally directed expansion force, both sided on the balls of the thumb of the affected hand. An expansion of some millimeters is targeted, which is comparable to a surgical intervention with an attained extension of approx. 3 to 4 mm. In the process the expansion force should preferably be horizontally transmitted within the area of the palm directly on the carpal ligament. This is realized by means of adhesive elements which can be pasted on the skin of the palms as sort of a plaster.

### DETAILED DESCRIPTION OF THE PREFERRED EMBODIMENTS

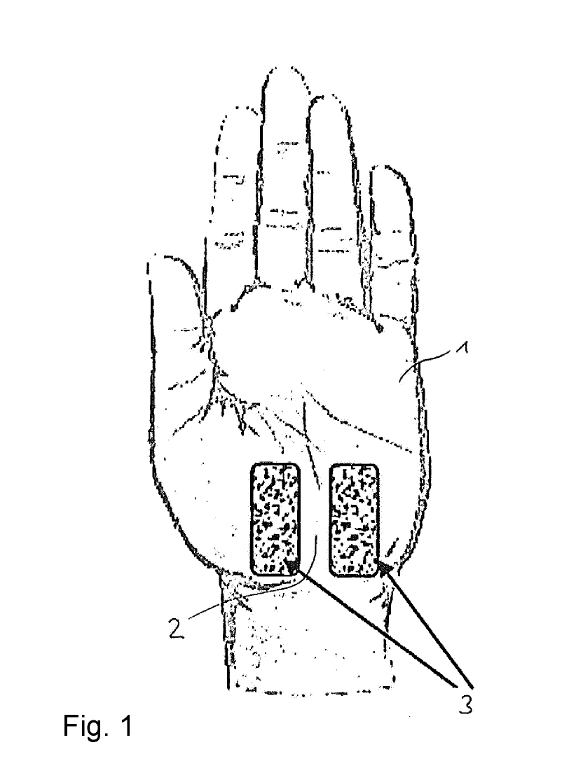

In FIG. 1 the inner side of a hand 1 is shown where laterally to the course of the carpal channel 2 two adhesive elements 3 are taped left and right-sided to the ball of the thumb. The adhesive elements 3 are adhesive pads which are provided with a skin-compatible, self-adhesive coat on the side of the part which is in direct contact with the skin. On the upper surface of the adhesive pads 3, the fluffy or the loop side of the Velcro™ is provided as shown. The adhesive pads are glued on the skin and allow a consistent and precise transmission of the expansion force from a corresponding expansion mechanism.

The adhesive pads are configured as disposable articles; however, they can stay on the hand for several days thus enabling a wide range of treatments. Whenever the adhesive pads are soiled or detached, they will be replaced by new ones.

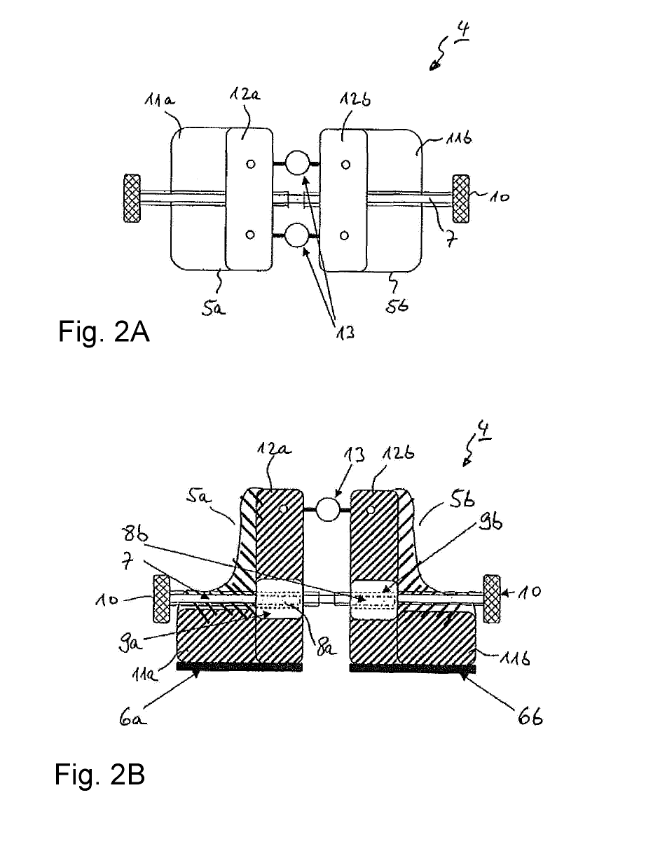

In FIGS. 2A and 2B, an expansion mechanism is shown, that is able to transfer an expansion force to the adhesive pads. This expansion mechanism contains two lateral shoes 5a, 5b, having on their reverse side in the area of the contact surface of the hand, the hook-side of the Velcro™ 6a, 6b. By means of the threaded spindle 7, the shoes 5a, 5b can be moved in the manner of a vice. The threaded spindle 7 of the demonstrated example is finished as an end-to-end thread bar having a left-hand thread on the left side 8a and a right-hand thread 8b on the right side. Accordingly the left shoe 5a has a threaded sleeve 9a with a left-hand thread, and the right shoe 5b has a threaded sleeve 9b with a right-hand thread. On both sides of the threaded spindle 7 a knurled bolt head is situated, in order to allow an easy bilateral operation.

On the lower part, both shoes 5a, 5b broaden to a base 11a, 11b building the contact surface to the hand. On the upper part, both shoes 5a, 5b extend in the form of blades 12a, 12b basically vertical to the contact surface of the hand 6a, 6b. The blades 12a, 12b are connected with each other via two hinges 13 at their top end. The joints/hinges 13 serve as compensation of the counter pressure by causing a basically lateral power transmission on the adhesive pads 3.

The mechanism to spread the shoes 5a, 5b can be varied in various ways. For example, the threaded spindle 7 can be separated in the middle and reconnected in a rotatable manner so that the threads on both sides can be produced as right-hand threads. In addition it would be possible to pass a threaded spindle only through one of the two shoes 5a, 5b, stabilizing itself with the second shoe and being rotatable there. At this point a multitude of different spreading methods as for example a lever mechanism, are possible too. As well, the shoes can be spread by means of an air cushion being situated between the two shoes which can be inflated by a manual pump. In order to secure a better hold of the expansion unit 4 on the adhesive pads, an additional support strap can be fixed which can be put over the back of the hand.

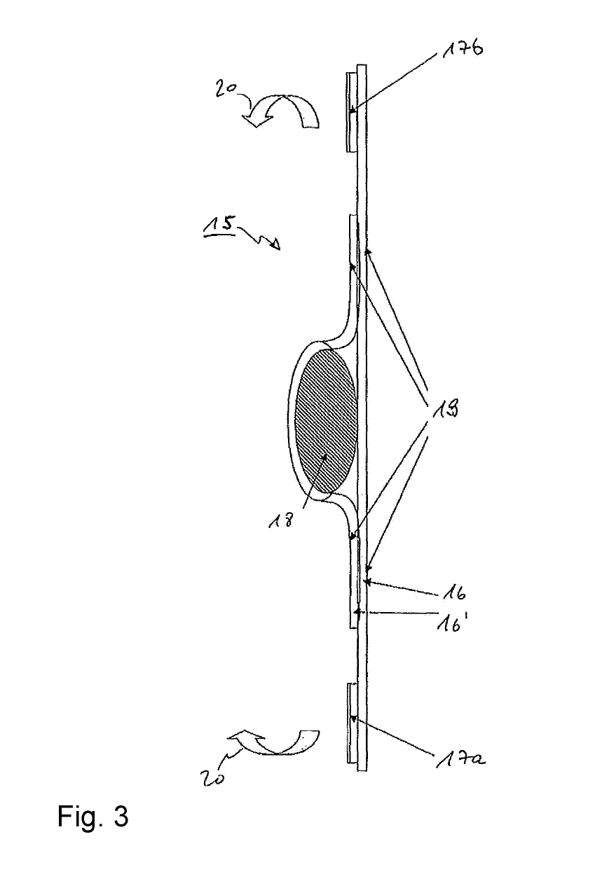

A second version for the expansion device is shown in FIG. 3. Here the expansion device 15 is designed in a bandage-style, comprising a stretch bandage 16 made out of an elastic textile fabric. On the inside of the stretch tape 16, the hook side of the Velcro™ fastener is sewn on either side.

Approximately in the middle and inside of the stretch tape 16, there is a pad 18 which is designed as elastic foam part. Thereby the pad 18 is sewn over a second layer of the elastic textile fabric 16′ on the inside of the stretch tape 16. The sewn area is schematically identified with the reference number 19.

The bandage 15 is put around the hand in the direction of the indicated arrows 20, in a way that the pad 18 finds its final position on the back of the hand. The Velcro™ fasteners 17a, 17b are fixed on the adhesive pads 3 which are pasted on the inside hand. Thus the Velcro™ fasteners easily allow the tension of the stretch tape as well as the regulation of the expansion force. Due to its elastic capacity, the pad 18 on the back of the hand provides sufficient back pull on the back of the hand. The pad 18 can additionally or alternatively contain an air buffer, which allows a regulation of the expansion force being transferred on the adhesive pads, by inflating it by means of a manual pump. Consequently the pad 18 supports the expansion of the carpal channel.

If required the elastic bandage 15 can be removed and reattached because of the Velcro™ adhesive pads. The material of the brace is similar to an elastic bandage and can be produced in different flexibilities. Therefore an accelerating treatment is possible according to the expansion progress. The expansion device can be individually adapted to the size of the wrist by means of the brace having different lengths. The fine adjustment will be realized by the Velcro™ fastener.

1. A treatment device for stretching the carpal tunnel (2) of the human hand (1), by comprising at least two adhesive elements (3) which are suitable for coming into contact with human skin and designed for application on [[the]] thenar and hypothenar eminences of a human hand (1) to be treated, and an expansion mechanism (4) that engages on the adhesive elements (3) or is connected to them and exerts a basically laterally directed expansion force on the adhesive elements (3).

2. The treatment device as claimed in claim 1, wherein the adhesive elements (3) are straps having a self-adhesive coat at least on one side.

3. The treatment device as claimed in claim 2, wherein the straps are provided, on a side opposite the self-adhesive coat, with a reversible quick-release fastener.

4. The treatment device as claimed in claim 1, wherein the expansion mechanism (4) comprises two shoes (5a, 5b) which are braceable against each other.

5. The treatment device as claimed in claim 4, wherein the shoes (5a, 5b) are supported against each other via at least one threaded spindle (7).

6. The treatment device as claimed in claim 5, wherein the shoes (5a, 5b) are interconnected, above the threaded spindle (7), by at least one joint (13).

7. The treatment device as claimed in claim 5, wherein the shoes (5a, 5b) each have, on an underside thereof, a contact surface (6a, 6b) which carries a reversible quick-release fastener.

8. The treatment device as claimed in claim 7, wherein an outline of the contact surface (6a, 6b) is adapted individually to a shape of eminences of the hand (1) to be treated.

9. The treatment device as claimed in claim 1, wherein the expansion mechanism is comprises a bandage and an elastic stretch belt (16), which is constructed in such a way that it is put around a back of the hand (1) to be treated in order to transmit the expansion force to the adhesive elements (3).

10. The treatment device as claimed in claim 9, wherein the stretch belt (16) has an inner side and an outer side, and in each case one reversible quick-release fastener (17a, 17b), is provided at ends on the inner side.

11. The treatment device as claimed in claim 9, wherein the stretch belt (16) carries a padding (18) approximately in a middle thereof.

12. The treatment device as claimed in claim 11, wherein the padding (18) is an elastic pressure padding.

13. The treatment device as claimed in claim 11, wherein the padding (18) is an elastic foam body.

14. The treatment device as claimed in claim 11, wherein the padding (18) contains an air cushion, which is inflatable by a manual pump.

15. The treatment device as claimed in claim 1, wherein the two adhesive elements (3), are designed to be adhesively bonded, laterally with respect to the carpal tunnel (2), to eminences of a human hand to be treated, and the expansion mechanism (4) is designed to be connected to the adhesive elements (3) in order to transmit the laterally directed expansion force targetedly and directly onto the eminences, laterally with respect to the carpal tunnel (2).
